(19) United States
(12) Patent Application Publication
(10) Pub. No.: US 2023/0151343 A1
(43) Pub. Date: May 18, 2023

(54) GENOME EDITING USING CAS9 OR CAS9 VARIANT

(71) Applicant: INSTITUTE FOR BASIC SCIENCE, Daejeon (KR)

(72) Inventors: Jin-Soo KIM, Seoul (KR); Kayeong LIM, Seoul (KR); Jaesuk LEE, Seoul (KR)

(21) Appl. No.: 17/919,588

(22) PCT Filed: Apr. 26, 2021
(86) PCT No.: PCT/KR2021/005244

§ 371 Date: Oct. 18, 2022

(30) Foreign Application Priority Data
Apr. 24, 2020 (KR) 10-2020-0049972

(51) Int. Cl. C12N 9/22

(52) U.S. Cl. CPC C12N 9/22

(57) ABSTRACT

The present invention relates to a Cas9 variant or a nucleic acid encoding the same, a composition for editing a genome using Cas9 or a Cas9 variant or a nucleic acid encoding the same, and a method of editing a genome using the composition. Specifically, the present invention relates to a composition for editing a genome with excellent efficiency while reducing unwanted insertions/deletions (indels) by using a prime editing nuclease or a variant thereof, for example, Cas9 or a Cas9 variant or a nucleic acid encoding the same, and a method of editing a genome using the composition.

### TECHNICAL FIELD

The present invention relates to a Cas9 variant or a nucleic acid encoding the same, a composition for editing a genome using Cas9 or a Cas9 variant or a nucleic acid encoding the same, and a method of editing a genome using the composition. Specifically, the present invention relates to a composition for editing a genome with excellent efficiency while reducing unwanted insertions/deletions (indels) by using a prime editing nuclease or a variant thereof, for example, Cas9 or a Cas9 variant or a nucleic acid encoding the same, and a method of editing a genome using the composition.

### BACKGROUND ART

To overcome flexibility and precision limitations shown in gene editing by CRISPR, which includes a molecular complex comprising a guide DNA that recognizes a specific position in a genome and a Cas9 enzyme that cuts the DNA double helix, improved genome editing methods have been reported.

Specifically, there has been reported a method for editing a genome using a prime editor protein complex composed of nickase Cas9 (H840A) and M-MLV reverse transcriptase, in which the nickase Cas9 is modified to cut only one strand of DNA, the reverse transcriptase copies an RNA template to make new DNA, and prime editing guide RNA (pegRNA) directs the prime editor protein complex to the target site (Anzalone A V, Randolph P B, Davis J R et al., “Search-and-replace genome editing without double-strand breaks or donor DNA,” Nature. 2019 Oct 21).

Under this technical background, the inventors of this application have found that a prime editor protein containing nuclease which is not nickase can also induce prime editing, and when a nickase that cuts a non-target strand is generated by introducing mutation into nickase or deleting some amino acid residues, it is possible to perform desired gene editing with excellent efficiency while significantly reducing unwanted insertions/deletions that may occur when repairing DSBs, and the nickase can be delivered via a size-restricted adeno-associated virus (AAV) vector, thereby completing the present invention.

### SUMMARY OF THE INVENTION

An object of the present invention is to provide a composition for editing a genome using Cas9 or a Cas9 variant, and a genome editing method using the same.

Another object of the present invention is to provide a nuclease variant, for example, a Cas9 variant.

To achieve the above objects, the present invention provides a nuclease variant in which one or more amino acids selected from the group consisting of D839, H840, N854 and N863 in the sequence of SEQ ID NO: 1 are substituted with other amino acid(s), or a nucleic acid encoding the nuclease variant.

The present invention also provides a nuclease variant containing a deletion of one or more amino acid residues selected from the group consisting of the following amino acid residues in a sequence selected from the group consisting of SEQ ID NOs: 1 to 15, or a nucleic acid encoding the nuclease variant:

a deletion of one or more amino acid residues at positions 824 to 874 in a sequence selected from the group consisting of SEQ ID NOs: 1 to 15;

a deletion of one or more amino acid residues at positions 792 to 897 in a sequence selected from the group consisting of SEQ ID NOs: 1 to 15;

a deletion of one or more amino acid residues at positions 786 to 885 in a sequence selected from the group consisting of SEQ ID NOs: 1 to 15; and

a deletion of one or more amino acid residues at positions 765 to 908 in a sequence selected from the group consisting of SEQ ID NOs: 1 to 15.

The present invention also provides a composition for genome editing containing: (1) a prime editor protein comprising a nuclease or a variant thereof and a reverse transcriptase, or a nucleic acid encoding the prime editor protein; and (2) a prime editing guide RNA (pegRNA) comprising a binding site, which binds to a genome to be edited, and an editing sequence.

The present invention provides the use of a composition for use in the manufacture of an agent for genome editing, wherein the composition contains: (1) a prime editor protein comprising a nuclease or a variant thereof and a reverse transcriptase, or a nucleic acid encoding the prime editor protein; and (2) a prime editing guide RNA (pegRNA) comprising a binding site, which binds to a genome to be edited, and an editing sequence.

The present invention also provides a genome editing method comprising a step of treating a subject with a composition for genome editing, the composition containing: (1) a prime editor protein comprising a nuclease or a variant thereof and a reverse transcriptase, or a nucleic acid encoding the prime editor protein; and (2) a prime editing guide RNA (pegRNA) comprising a binding site, which binds to a genome to be edited, and an editing sequence.

### BRIEF DESCRIPTION OF DRAWINGS

FIG. 1 shows the predicted and experimental results for the cleavage of a target sequence using Cas9, nCas9-D10A and nCas9-H840A.

(a) Schematic overview of Digenome-seq. Information on a target sequence; predicted cleavage positions upon cleavage with Cas9, nCas9-D10A and nCas9-H840A in vitro (red arrowhead: cleavage position, blue: PAM sequence); results predicted when examining whole genome sequencing (WGS) data by IGV (red: forward strand, blue: reverse strand). (b) Results of gDNA cleavage in vitro. gDNA of HAP1 cells was treated with each of Cas9 variants at 37° C. for 16 hours, and WGS results were checked. Cas9 and nCas9-D10A showed the same cleavage pattern as expected, but in the case of nCas9-H840A, partial cleavage occurred in the target strand, contrary to expectations. (c) In vitro plasmid cleavage experiment. In order to confirm the cleavage experiment performed on gDNA again, an in vitro cleavage experiment was performed using a plasmid. Upon electrophoresis, a supercoiled plasmid remains in a linear form when both strands are cleaved and in an open circular form when one strand is cleaved. With a 6,030-bp plasmid, an open circular plasmid and a linear plasmid for comparison were constructed using Nt.BbvCI enzyme that cleaves one strand and SpeI enzyme that cleaves both strands, respectively. Thereafter, the plasmids were treated with each of Cas9, nCas9-D10A and nCas9-H840A, and the form of each plasmid was observed. It was confirmed that, when the plasmids were treated with Cas9, most of the plasmids were cleaved in both strands and remained in a linear form, and when the plasmids were treated with nCas9-D10A, most of the plasmids were cleaved in one strand and remained in an open circular form. However, it was confirmed that, when nCas9-H840A was used, more linear plasmids appeared than when nCas9-D10A was used. As a result of measuring the intensities of the bands using ImageJ software and obtaining relative linear band intensity values, it was observed that the relative band intensity values were linear 16.0% for nCas9-D10A and 43.3% for nCas9-H840A.

FIG. 2 shows the results of constructing a Cas9 variant and examining the frequency of unwanted insertions/deletions (indels) that can be introduced using the Cas9 variant.

(a) As nuclease domains of SpCas9, an HNH domain and a RuvC domain exist, which cleave target DNA and non-target DNA, respectively. When a mutation is introduced into the HNH domain or RuvC domain of Cas9, it is possible to produce a Cas9 nickase that can cut only one strand. As Cas9 nickase, a form in which D10A mutation is introduced into the RuvC domain or a form in which H840A or N863A mutation is introduced into the HNH domain is mainly used. In this study, mutations were introduced at positions D839, H840, N854 and N863 in the HNH domain, which are involved in DNA cleavage, to create a Cas9 nickase that can completely cut only a non-target strand.

(b) To examine the frequency of unwanted indels (insertions and deletions) that can be introduced in cells by nickase Cas9 (nCas9), nCas9 was delivered into HEK293T cells together with plasmids expressing sgRNAs targeting various genes. Next, the cell DNA was isolated and analyzed by targeted deep-sequencing. As a result, an indel frequency of 0.035 to 15% (2.5% on average) was shown by HNHv1(Cas9-H840A) which is mainly used in the prior art. To reduce the indels, Cas9 variants having mutations of combinations of D839A, H840A, N854A and N863A in the Cas9 HNH domain were produced and used. As a result, it could be confirmed that the frequency of unwanted indels was reduced to less than 1% on average upon the use of various variants (HNHv5(H840A/N863A), HNHv7(H840A/N854A), HNHv9(N863A/N854A), HNHv11(H840A/N863A/N854A), HNHv12(H840A/D839A/N854A), HNHv13(N863A/D839A/N854A), and HNHv14(H840A/N863A/D839A/N854A)).

(c) In order confirm whether the reduction in the frequency of unwanted indels as shown in the previous experiment is because the Cas9 variant is 1) a nickase form that accurately cuts only one strand or 2) a catalytically dead Cas9 form that lacks the activity of Cas9 and does not cut both strands, a double nicking experiment (an experiment using two sgRNAs that cut different strands) was conducted. In the case of 1), upon treatment with sgRNA-A or sgRNA-1, indels will not be observed, and upon treatment with both sgRNA-A and sgRNA-1, both strands will be cut (DNA double strand breaks) and indels will be observed. In case of 2), indels will not be observed upon treatment with sgRNA-A, sgRNA-1, or sgRNA-A+sgRNA-1. As a result of confirming this prediction experimentally for two target sites, it could be confirmed that HNHv7, HNHv11, HNHv12 and HNHv14 showed an indel frequency of 1% or less upon treatment with sgRNA-A, sgRNA-1, or sgRNA-A+sgRNA-1, suggesting that they are catalytically dead Cas9s that have lost almost all activity. On the other hand, it could be confirmed that HNHv5, HNHv9 and HNHv13 showed an indel frequency of 1% or less upon treatment with sgRNA-A or sgRNA-1, but showed an indel frequency of 1% or more upon treatment with sgRNA-A and sgRNA-1, suggesting that they are in the form of a Cas9 nickase that cuts one strand.

FIG. 3 shows the results of examining changes in cleavage patterns in an in vitro experiment.

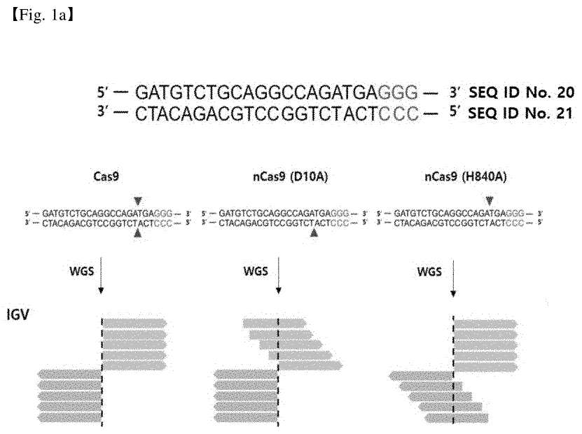

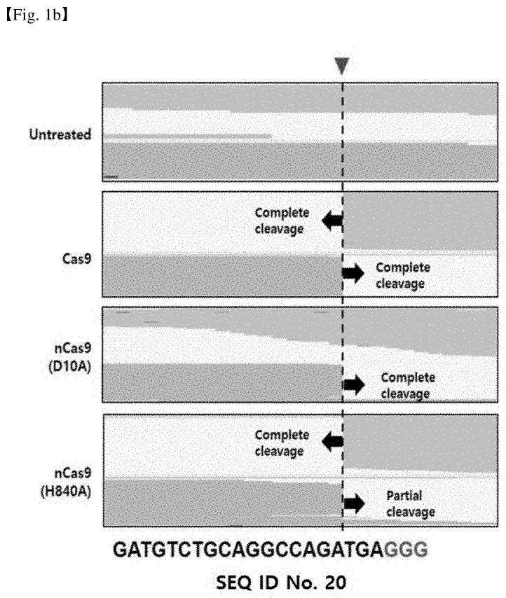

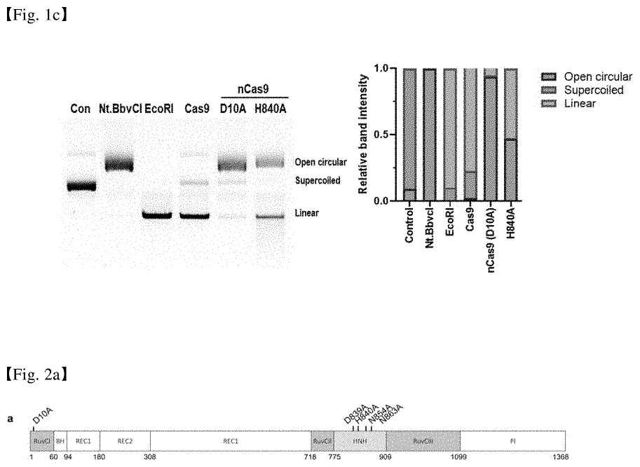

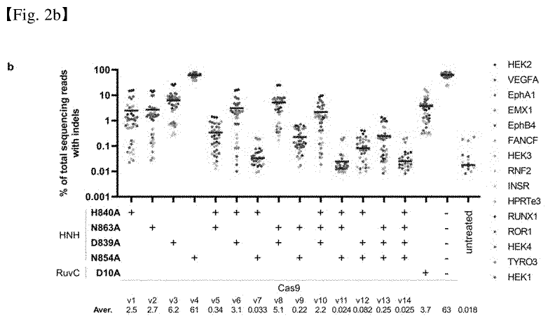

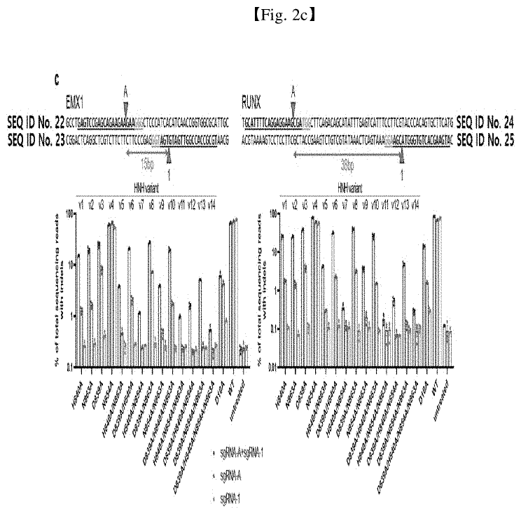

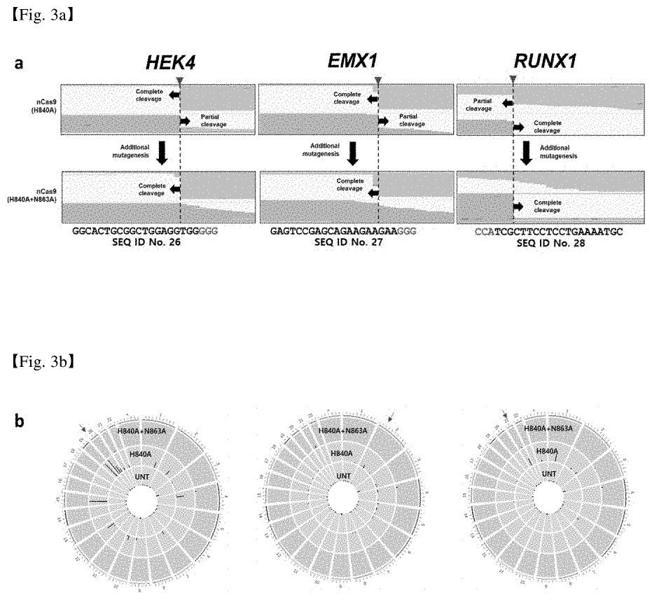

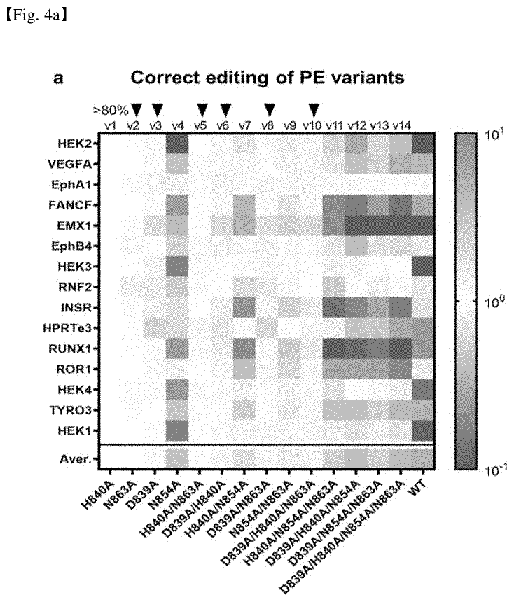

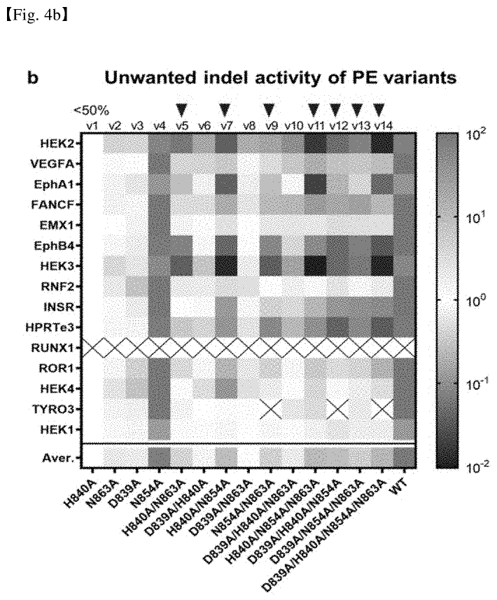

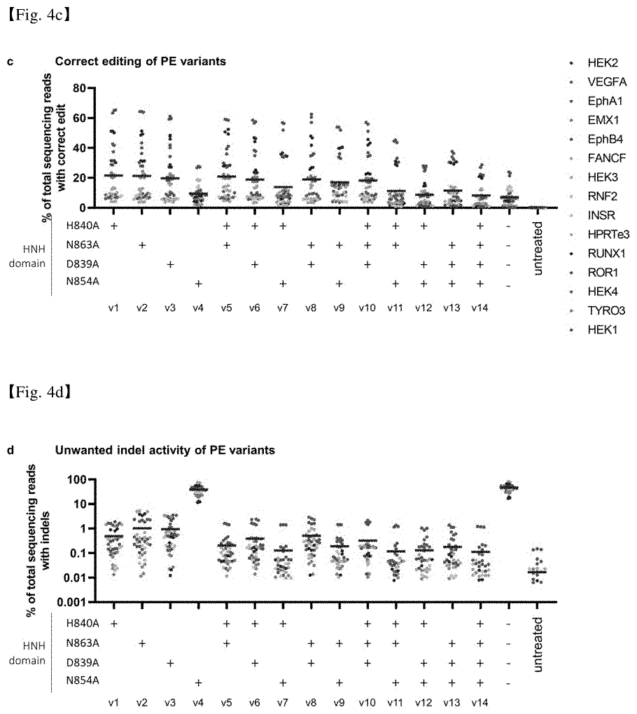

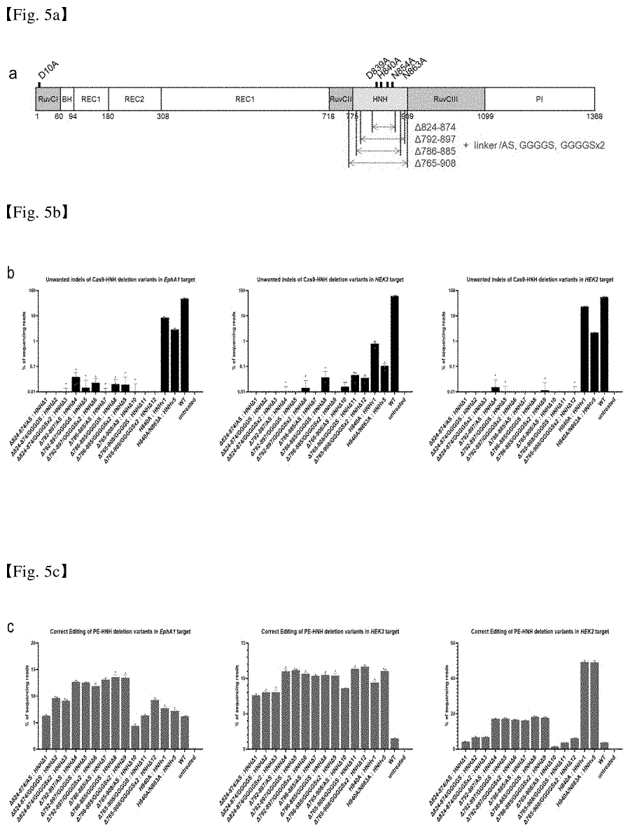

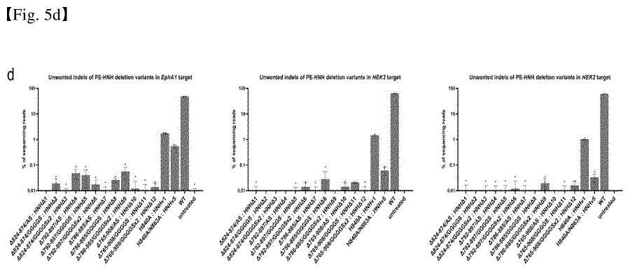

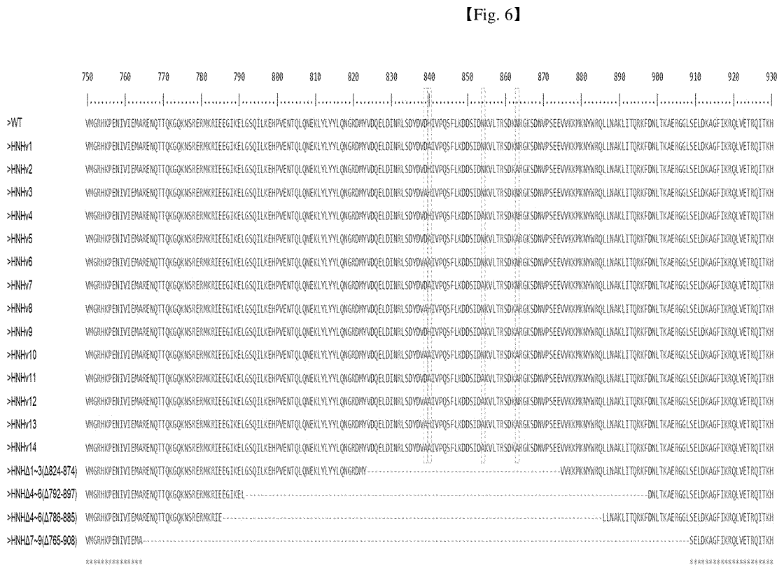

(a) gDNA of isolated cells was treated with each of nCas9-H840A and nCas9-H840A/N863A, and changes in the cleavage pattern of the gDNA were examined by WGS. As a result of targeting three different sites (HEK4, EMX1 and RUNX1), it could be confirmed that nCas9-H840A induced partial double strand cleavage, whereas, upon treatment with nCas9-H840A/N863A, cleavage of only a desired non-target strand occurred. (b) Pattern changes in the whole genome by Digenome sequencing. Digenome sequencing is one of the methods that can detect double-strand breaks in the whole genome. The patterns of double-strand breaks appearing in the whole genome were compared through digenome sequencing, and the results were displayed by Circos plots. When three different sites (HEK4, EMX1 and RUNX1) were treated with nCas9-H840A, double-strand breaks were observed at the target sites and off-target sites. On the other hand, upon treatment with nCas9-H840A/N863A, double-stranded break could not be observed at the target sites, and it could be confirmed that double strand breaks at off-target sites disappeared or the percentage thereof was significantly reduced. Thereby, it was confirmed from the in vitro experimental results that Cas0-H840A/N863A is a nickase Cas9 form that can cut only one strand of DNA, as shown in FIG. 1.

FIG. 4 shows the results of measuring the efficiency of gene editing and the frequency of unwanted indels upon the use of the prime editor proteins according to the present invention.

A prime editor (PE) composed of nCas9 and MMLV reverse transcriptase was delivered to cells together with pegRNA capable of inducing a mutation to be introduced, and DNA was analyzed by targeted deep-sequencing, and the efficiency of desired gene editing (correct editing) (a) and the unwanted indel activity (b) were measured. The indicated values were all normalized to 1, which is a value for conventional PEv1(PE-H840A). When the efficiency of desired gene editing is higher than 1, it is shown in pink, and when the efficiency of desired gene editing is lower than 1, it is shown in green. When the unwanted indel activity is higher than 1, it is shown in red, and when the unwanted indel activity is lower than 1, it is shown in blue. (c, d) non-normalized NGS data.

(a, c) When PE variants were prepared using the Cas9 variants used in FIG. 1 and were tested, it can be seen that, in the case of PE-HNHv3, PE-HNHv5, PE-HNHv6 and PE-HNHv8, PE-HNHv10 in comparison with conventional PE-HNHv1(PE2-H840A), the correct editing efficiency was retained. (Since it is preferable that the desired editing efficiency is not reduced, the values in FIG. 4a should not be green.)

(b, d) It can be seen that the frequency of unwanted indels introduced by PE-HNHv1 was reduced to less than half when PE-HNHv5, PE-HNHv7, PE-HNHv9, PE-HNHv11, PE-HNHv12, PE-HNHv13 or PE-HNHv14 was used. (Since it is preferable that the frequency of unwanted indels be reduced, it is preferable that the values in FIG. 4b are blue.)

Thereby, it was confirmed that, when PE-HNHv5 (PE2-H840A/N863A) among the HNH domain variants of PE obtained by introducing mutations into the HNH domain was used, the frequency of unwanted indels was reduced compared to when the conventional PE-HNHv1(PE2-H840A) was used, and the desired genome editing efficiency was retained. In addition, it could be confirmed that, even when PE2-Cas9-WT composed of a Cas9 nuclease form (the form in which the conventional H840A mutation was removed) was used, the desired genome editing efficiency of 13.0% on average was obtainable, and in targets in which the efficiency of PE is very low, the correct editing efficiency was sometimes increased when Cas9 nuclease was used (the pink color observed in the PE-Cas9-WT portion in FIG. 4a).

FIG. 5 shows the results of measuring the efficiency of gene editing and the frequency of unwanted indels upon the use of Cas9 variants containing a deletion of additional amino acid residues.

(a) To further reduce unwanted indel mutations, HNH deletion variants (HNHΔ1 to 12) were prepared by deleting a portion of the HNH domain of Cas9 and then linking with linkers of various lengths (amino acid sequences: AS, GGGGS, and GGGGSGGGGS).

(b) The frequency of unwanted indels introduced into various HNH deletion variants (HNHΔ1 to 12) obtained by introducing HNH deletion into Cas9 was measured at three different target sites. It was confirmed that Cas9-HNHΔ1 to 12 introduced indels with much lower efficiency than the conventional Cas9-H840A and the Cas9-HNHv5(Cas9-H840A/N863A) identified in the previous experiment.

(c) PE variants were prepared using various HNH deletion variants (HNHΔ1 to 12), and cells were treated with the PE variants. Then, the efficiency of correct genome editing was measured by targeted deep-sequencing. As a result, it was confirmed that, in the case of PE-HNHΔ4 to 9, desired editing occurred well with similar efficiency or half efficiency compared to that in the case of the conventional PE or PE-HNHv5.

(d) The frequency of unwanted indels that can be introduced by PE-HNH deletion variants (HNHΔ1 to 12) was measured. It was confirmed that, in the case of PE-HNHΔ4 to 9, unwanted indels were significantly reduced. In addition, it was confirmed that the frequency of unwanted indels was reduced compared to when the previous HNH point mutation variants (HNHv1 to 14) were used. Thereby, it was confirmed that, when PEs without the 792-897 amino acid portion or 786-885 amino acid portion of Cas9 are used, the introduction of unwanted indels may be reduced and correct gene editing may occur well. As a result, even if about 100 amino acids in the Cas9 sequence are deleted, the gene editing function of PEs can be performed well, and the sizes of Cas9 and PE proteins also become smaller.

FIG. 6 shows the results of sequence comparison between wild-type Cas9 and Cas9 variants.

### DETAILED DESCRIPTION OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

Unwanted indel mutations are introduced because the H840A Cas9 nickase constituting PE is not a complete nickase. To overcome this problem, variants were prepared by variously modifying the HNH domain of Cas9. As a result of measuring the frequency of indels and the efficiency of correct editing upon the use of various Cas9 variants, including “point mutation variants” prepared by introducing point mutations into the HNH domain and “deletion mutation variants” prepared by deleting a portion of the HNH domain, as well as PE variants, it was confirmed that the use of specific variants (HNHv5(H840A/N863A), HNHΔ4-6(Δ792-897), HNH7-9(Δ786-885)) could induce desired gene editing without introduction of unwanted indels. The use of these variants may induce correct gene editing without introduction of unwanted mutations, and moreover, the deletion variant has the advantage of reducing the size of the protein by about 100 amino acids.

In a specific embodiment according to the present invention, the use of a prime editor protein composed of Cas9 nuclease (Cas9 WT) may also induce prime editing.

In addition, when various mutations were introduced into the HNH domain of Cas9, incomplete nickase (that cuts a non-target strand and some target-strands) could be made into a nickase form that cuts only the non-target strand. In addition, in a prime editing method using a prime editor protein having Cas9 nickase as a component, the use of Cas9 nickase variants (HNHv5(H840A/N863A), HNHΔ4-6(Δ792-897), or HNH7-9(Δ786-885)) that cut only a non-target strand may overcome the problem associated with the introduction of unwanted indels and induce desired correct gene editing. In particular, the deletion variants (HNHΔ4-6(Δ792-897), and HNH7-9(Δ786-885)) have an advantage in that they work well even if the size of the Cas9 protein is reduced by about 100 amino acids. If a size-restricted adeno-associated virus (AAV) vector is used, it is much more advantageous to use a deletion variant that has a small size and reduces unwanted indels.

Therefore, in one aspect, the present invention is directed to a nuclease variant in which one or more amino acids selected from the group consisting of D839, H840, N854 and N863 in the sequence of SEQ ID NO: 1 are substituted with other amino acid(s), or a nucleic acid encoding the nuclease variant.

Another aspect of the present invention is directed to a nuclease variant containing a deletion of one or more amino acid residues at positions 765 to 908 in a sequence selected from the group consisting of SEQ ID NOs: 1 to 15, or a nucleic acid encoding the nuclease variant.

Specifically, the present invention may include a nuclease variant containing a deletion of one or more amino acid residues selected from the group consisting of the following amino acid residues in a sequence selected from the group consisting of SEQ ID NOs: 1 to 15, or a nucleic acid encoding the nuclease variant:

a deletion of one or more amino acid residues at positions 824 to 874 in a sequence selected from the group consisting of SEQ ID NOs: 1 to 15;

a deletion of one or more amino acid residues at positions 792 to 897 in a sequence selected from the group consisting of SEQ ID NOs: 1 to 15;

a deletion of one or more amino acid residues at positions 786 to 885 in a sequence selected from the group consisting of SEQ ID NOs: 1 to 15; and

a deletion of one or more amino acid residues at positions 765 to 908 in a sequence selected from the group consisting of SEQ ID NOs: 1 to 15.

The present invention is also directed to a composition for genome editing containing: (1) a prime editor protein comprising a nuclease or a variant thereof and a reverse transcriptase, or a nucleic acid encoding the prime editor protein; and (2) a prime editing guide RNA (pegRNA) comprising a binding site, which binds to a genome to be edited, and an editing sequence.

The nuclease may be target-specific and may be, for example, ZNFN (zinc finger nuclease), TALEN (transcriptional activator-like effector nuclease) or Cas protein, without being limited thereto. The Cas protein may be Cas1, Cas1B, Cast, Cas3, Cas4, Cas5, Cas6, Cas7, Cas8, Cas9, Cas10, Cas12a, Cas12b, Cas12c, Cas12d, Cas12e, Cas12g, Cas12h, Cas12i, Cas12j, Cas13a, Cas13b, Cas13c, Cas13d, Cas14, Csy1, Csy2, Csy3, Cse1, Cse2, Csc1, Csc2, Csa5, Csn2, CsMT2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3 or Csf4 endonuclease, particularly Cas9, without being limited thereto.

The Cas protein is a major protein component of the CRISPR/Cas system, and is a protein capable of forming an activated endonuclease or nickase. The Cas protein may be, for example, derived or simply isolated from a Cas protein ortholog-containing microorganism selected from the group consisting of Corynebacter, Sutterella, Legionella, Treponema, Filifactor, Eubacterium, Streptococcus (Streptococcus pyogenes), Lactobacillus, Mycoplasma, Bacteroides, Flavivola, Flavobacterium, Azospirillum, Gluconacetobacter, Neisseria, Roseburia, Parvibaculum, Staphylococcus (Staphylococcus aureus), Nitratifractor, Corynebacterium and Campylobacter. Alternatively, the Cas protein may be a recombinant protein.

The Cas9 sequence can be found in a known database such as GenBank of NCBI (National Center for Biotechnology Information). The Cas9 may comprise, for example, the amino acid sequence of SEQ ID NO: 1.

The target-specific nuclease may be may be a microorganism-derived protein or an artificial or non-naturally occurring protein obtained by a recombinant or synthesis method. In one embodiment, the target-specific nuclease (e.g., Cas9, Cpf1, etc.) may be a recombinant protein produced from recombinant DNA. As used herein, the term “recombinant DNA (rDNA)” refers to a DNA molecule artificially made by genetic recombination, such as molecular cloning, to include therein heterogenous or homogenous genetic materials derived from various organisms. For instance, when a target-specific nuclease is produced in vivo or in vitro by expressing recombinant DNA in an appropriate organism, the recombinant DNA may have a nucleotide sequence reconstituted with codons selected from among codons encoding the protein of interest in order to be optimal for expression in the organism.

The nuclease may be a mutated target-specific nuclease. The term “mutated target-specific endonuclease” may refer to a target-specific nuclease that lacks the endonuclease activity of cleaving a DNA duplex. For example, the mutated target-specific nuclease may be one that lacks endonuclease activity, but retains nickase activity. Through the nickase, a nick may be introduced into any one of two strands.

The nuclease variant may be, for example, a Cas9 variant. The nuclease domain of Cas9 has an HNH domain and a RuvC domain, which can cut target DNA and non-target DNA, respectively. When a mutation is introduced in the HNH domain or RuvC domain of Cas9, it is possible to produce a Cas9 nickase that can cut only one strand.

In one embodiment, the nuclease variant may be a nuclease variant in which one or more amino acids selected from the group consisting of D839, H840, N854 and N863 in the sequence of SEQ ID NO: 1, which is the amino acid sequence of Cas9, are substituted with other amino acid(s).

Therefore, in another aspect, the present invention is directed to a nuclease variant in which one or more amino acids selected from the group consisting of D839, H840, N854 and N863 in the sequence of SEQ ID NO: 1 are substituted with other amino acid(s).

Specifically, the nuclease variant may contain one or more mutations selected from the group consisting of the following mutations:

a substitution of alanine for D839 in the sequence of SEQ ID NO: 1;

a substitution of alanine for H840 in the sequence of SEQ ID NO: 1;

a substitution of alanine for N854 in the sequence of SEQ ID NO: 1; and

a substitution of alanine for N863 in the sequence of SEQ ID NO: 1.

In one specific example of the present invention, a Cas9 variant in which one or more amino acids selected from the group consisting of D839, H840, N854 and N863 in the HNH domain of Cas9 are substituted with other amino acid(s) was produced. The Cas9 variant was named HNHv5(H840A/N863A), HNHv7(H840A/N854A), HNHv9(N863A/N854A), HNHv11(H840A/N863A/N854A), HNHv12(H840A/D839A/N854A), HNHv13(N863A/D839A/N854A), or HNHv14(H840A/N863A/D839A/N854A). It could be confirmed that, when a Cas9 variant of HNHv5(H840A/N863A), HNHv7(H840A/N854A), HNHv9(N863A/N854A), HNHv11(H840A/N863A/N854A), HNHv12(H840A/D839A/N854A), HNHv13(N863A/D839A/N854A), or HNHv14(H840A/N863A/D839A/N854A) was used, the frequency of unwanted indels was reduced to 1% or less on average.

In a specific embodiment according to the present invention, the present invention may include a nuclease variant comprising a sequence selected from the group consisting of SEQ ID NOs: 2 to 15.

| SEQ ID NO | Name |
|---|---|
| SEQ ID NO: 2 | HNHv1(H840A) |
| SEQ ID NO: 3 | HNHv2(N863A) |
| SEQ ID NO: 4 | HNHv3(D839A) |
| SEQ ID NO: 5 | HNHv4(N854A) |
| SEQ ID NO: 6 | HNHv5(H840A/N863A) |
| SEQ ID NO: 7 | HNHv6(H840A/D839A) |
| SEQ ID NO: 8 | HNHv7(H840A/N854A) |
| SEQ ID NO: 9 | HNHv8(N863A/D839A) |
| SEQ ID NO: 10 | HNHv9(N863A/N854A) |
| SEQ ID NO: 11 | HNHv10(H840A/N863A/D839A) |
| SEQ ID NO: 12 | HNHv11(H840A/N863A/N854A) |
| SEQ ID NO: 13 | HNHv12(H840A/D839A/N854A) |
| SEQ ID NO: 14 | HNHv13(N863A/D839A/N854A) |
| SEQ ID NO: 15 | HNHv14(H840A/N863A/D839A/N854A) |

In particular, it was confirmed that, when PE-HNHv5(PE2-H840A/N863A) among the HNH domain variants of prime editor protein obtained by introducing mutations into the HNH domain was used, the frequency of unwanted indels was reduced compared to when the conventionally known PE-HNHv1(PE2-H840A) was used, and the desired genome editing efficiency was retained.

In addition, it could be confirmed that, even when PE2-Cas9-WT composed of the Cas9 nuclease form (the form in which the existing H840A mutation was removed) was used, the desired genome editing efficiency was obtainable, and in targets in which the efficiency of PE is very low, the correct editing efficiency increased.

In still another aspect, the nuclease variant may contain a deletion of nuclease amino acid residue(s). The nuclease variant contains a deletion of one or more amino acid residues at positions 765 to 908 in any one sequence selected from the group consisting of SEQ ID NOs: 1 to 15. Specifically, the nuclease variant may contain a deletion of one or more amino acid residues selected from the group consisting of the following amino acid residues:

a deletion of one or more amino acid residues at positions 824 to 874 in any one sequence selected from the group consisting of SEQ ID NOs: 1 to 15;

a deletion of one or more amino acid residues at positions 792 to 897 in any one sequence selected from the group consisting of SEQ ID NOs: 1 to 15;

a deletion of one or more amino acid residues at positions 786 to 885 in any one sequence selected from the group consisting of SEQ ID NOs: 1 to 15; and

a deletion of one or more amino acid residues at positions 765 to 908 in any one sequence selected from the group consisting of SEQ ID NOs: 1 to 15.

Specifically, the nuclease variant may contain deletions in the HNH domain of Cas9, for example, amino acid deletions (HNHΔ1, HNHΔ2 and HNHΔ3) at positions 824 to 874, amino acid deletions (HNHΔ4, HNHΔ5 and HNHΔ6) at positions 792 to 897, amino acid deletions (HNHΔ7, HNHΔ8 and HNHΔ9) at positions 786 to 885, or amino acid deletions (HNHΔ10, HNHΔ11 and HNHΔ12) at positions 765 to 908.

| SEQ ID NO | Name |
|---|---|
| SEQ ID NO: 16 | HNHΔ1-3(Δ824-874) |
| SEQ ID NO: 17 | HNHΔ4-6(Δ792-897) |
| SEQ ID NO: 18 | HNHΔ4-6(Δ786-885) |
| SEQ ID NO: 19 | HNHΔ7-9(Δ765-908) |

Prime editor protein variants were prepared using various HNH deletion variants (HNHΔ1 to 12) described above, and cells were treated with the variants. Then, the efficiency of genome editing was measured by targeted deep-sequencing. As a result, it was confirmed that, in the case of PE-HNHΔ4 to 9, desired editing occurred well with similar efficiency or half efficiency compared to that in the case of the conventional PE or PE-HNHv5. However, it was confirmed that unwanted indels were significantly reduced in the case of PE-HNHΔ4 to 9.

In some cases, the composition may further contain a peptide linker at the C-terminus of the amino acid at position 823, the C-terminus of the amino acid at position 791, the C-terminus of the amino acid at position 785, or the C-terminus of the amino acid at position 764, instead of a deletion of amino acids at positions 824 to 874, positions 792 to 897, positions 786 to 885, or positions 765 to 908 in any one sequence selected from the group consisting of SEQ ID NOs: 1 to 15.

The peptide linker may be about 2 to 25 aa in length. For example, the peptide linker may comprise amino acids such as alanine, glycine and/or serine, without being limited thereto.

The linker may comprise, for example, (AnS)m (where n and m are each 1 to 10), (GS)n, (GGS)n, (GSGGS)n, or (GnS)m (where n and m are each 1 to 10). In particular, the linker may be, for example, (AnS)m or (GnS)m (where n and m are each 1 to 10). Specifically, the linker may be (AnS)m (where n=1 and m=1), or (GnS)m (where n=4 and m=1 or 2), that is, G₄S or (G₄S)₂.

The prime editing guide RNA comprises an editing sequence and functions as a reverse transcriptase template. The reverse transcriptase (RT) is an RNA-dependent DNA polymerase capable of synthesizing a DNA strand (i.e., complementary DNA, cDNA) using a reverse transcriptase template. Examples of the reverse transcriptase include, but are not limited to, M-MLV (Moloney murine leukemia virus) reverse transcriptase or a variant thereof, for example, M-MLV-RT lacking RNase H activity, or an M-MLV variant (D200N, T306K, W313F, T330P, or L603W), bovine leukemia virus (BLV) RT or a variant thereof, Rous sarcoma virus (RSV) RT or a variant thereof, or avian myeloblastosis virus (AMV) RT or a variant thereof.

Specifically, the reverse transcriptase may be an M-MLV reverse transcriptase derived from M-MLV (Moloney murine leukemia virus) or a variant thereof, for example, an M-MLV variant (D200N, T306K, W313F, T330P, or L603W) comprising the sequence of SEQ ID NO: 29.

The nuclease or variant thereof and the reverse transcriptase may individually comprise each nuclease or a variant thereof and a reverse transcriptase, and may be included in the form of a fusion protein of the nuclease or variant thereof and the reverse transcriptase.

The prime editing guide RNA (pegRNA) or DNA encoding the same comprises a binding site, which binds to a genome to be edited, and an editing sequence.

The sequence including the editing sequence serves as a reverse transcriptase template. The reverse transcriptase template comprises a desired editing sequence and has homology to the genomic DNA locus. The editing sequence is a heterologous sequence and includes a target sequence to be edited in the genome.

The binding site may be arbitrarily located in the 5′ direction or 3′ direction of the reverse transcriptase template, and specifically, the binding site may be located in the 3′ direction of the reverse transcriptase template.

The binding site may comprise a sequence complementary to a genomic DNA strand nicked by a nuclease (e.g., nickase) or a variant thereof contained in the prime editor protein. The binding site may hybridize to a target site, thereby serving as a target site for the initiation of reverse transcriptase activity.

The binding site may contain 5 or more, 6 or more, 7 or more, 8 or more, 9 or more, 10 or more, 11 or more, 12 or more, 13 or more, 14 or more, 15 or more, 20 or more, or 25 or more nucleotides, which have at least 80%, for example, at least 85%, at least 90%, at least 95%, at least 97%, at least 98%, at least 99%, or 100% homology to the sequence of the target site.

The composition according to the present invention contains: (1) a prime editor protein comprising a nuclease or a variant thereof and a reverse transcriptase, or a nucleic acid encoding the prime editor protein; and (2) a prime editing guide RNA (pegRNA) comprising a binding site, which binds to a genome to be edited, and an editing sequence. In order to deliver components (1) and (2), a single delivery means or a plurality of delivery means may be used in combination in the same or different configurations.

Component (1) may be contained in a first delivery means, and component (2) may be contained in a second delivery means. Each of the delivery systems may be a viral delivery means, or one of the delivery systems may be a viral delivery means and the other may be a non-viral delivery means. Alternatively, the delivery systems may all be non-viral delivery means.

The nucleic acid may be an RNA sequence, a DNA sequence, or a combination thereof (RNA-DNA combination sequence). The prime editing guide RNA may comprise an RNA sequence of the guide RNA or a DNA sequence encoding the RNA sequence.

The DNA sequence encoding the prime editor protein (1) and the DNA sequence encoding the prime editing guide RNA (2) may be provided through a delivery means such as a vector. The DNA sequence encoding component (1) and the DNA sequence encoding component (2) may be placed on the same vector, so that they may be delivered simultaneously by the single vector. The DNA sequence encoding the prime editor protein (1) and the DNA sequence encoding the prime editing guide RNA (2) may be placed on different vectors and delivered by the vectors.

The composition according to the present invention may be delivered using a viral vector, for example, adeno-associated viral vector (AAV), adenoviral vector (AdV), lentiviral vector (LV) or retroviral vector (RV), as well as other viral vectors, for example, episomal vectors containing Simian virus 40 (SV40) ori, bovine papilloma virus (BPV) ori, or Epstein-Barr nuclear antigen (EBV) ori.

The vector may be delivered in vivo or into cells by a local injection method (e.g., direct injection into a lesion or target site), electroporation, lipofection, viral vector, nanoparticles, PTD (protein translocation domain) fusion protein method, or the like.

In some cases, the DNA sequence encoding the prime editing guide RNA (2) may be delivered by a vector. The prime editor protein (1) or an RNA sequence encoding the same may be delivered in the form of mRNA. The prime editor protein or mRNA may be delivered directly or delivered by a carrier.

In addition, the composition may contain the RNA sequence encoding the prime editor protein (1) and the prime editing guide RNA sequence (2). The mRNA encoding component (1) and the mRNA of component (2) may be delivered. The mRNAs may be delivered directly or delivered by a carrier.

Furthermore, an RNP (ribonucleoprotein) complex formed by assembling the prime editor protein (1) and the mRNA of the prime editing guide RNA (2) may be delivered. The RNP may be delivered directly or delivered by a carrier.

The RNP complex may be delivered into cells by various methods known in the art, such as microinjection, electroporation, DEAE-dextran treatment, lipofection, nanoparticle-mediated transfection, protein transduction domain-mediated introduction, and PEG-mediated transfection, without being limited thereto.

The carrier may comprise, for example, a cell penetrating peptide (CPP), nanoparticles, or a polymer, without being limited thereto. CPPs are short peptides that facilitate cellular uptake of a variety of molecular cargoes (from nanosized particles to small chemical molecules and large fragments of DNA). The cargo may comprise: (1) a prime editor protein or a nucleic acid encoding the same; and (2) prime editing guide RNA. The prime editor protein (1) or a nucleic acid encoding the same may be assembled through a chemical covalent bond or a non-covalent interaction. The prime editing guide RNA (2) or a polynucleotide encoding the same is complexed with CPP to form condensed, positively charged particles.

With respect to the nanoparticles, the composition according to the present invention may be delivered by polymer nanoparticles, metal nanoparticles, metal/inorganic nanoparticles, or lipid nanoparticles. The polymer nanoparticles may be, for example, DNA nanoclews or yarn-like DNA nanoparticles synthesized by rolling circle amplification. DNA nanoclews or yarn-like DNA nanoparticles were loaded with: (1) a prime editor protein or a nucleic acid encoding the same; and (2) a prime editing guide RNA, and coated with PEI to enhance the endosomal escape ability. This complex may bind to the cell membrane, may be internalized, and then may migrate to the nucleus through endosomal escape, allowing simultaneous delivery of (1) and (2).

With respect to the metal nanoparticles, (1) a prime editor protein or a nucleic acid encoding the same, and (2) a prime editing guide RNA may be linked to gold particles and complexed with a cationic endosomal disruptive polymer, followed by intracellular delivery. The cationic endosomal disruptive polymer may be, for example, polyethylene imine, poly(arginine), poly(lysine), poly(histidine), poly-[2-{(2-aminoethyl)amino}-ethyl-aspartamide] (pAsp(DET)), a block copolymer of polyethylene glycol) (PEG) and poly(arginine), a block copolymer of PEG and poly(lysine), or a block copolymer of PEG and poly{N—[N-(2-aminoethyl)-2-aminoethyl]aspartamide} (PEG-pAsp(DET)).

With respect to the metal/inorganic nanoparticles, (1) a prime editor protein or a nucleic acid encoding the same, and (2) a prime editing guide RNA may be encapsulated with, for example, ZIF-8 (zeolitic imidazolate framework-8), or a negatively charged RNP may be encapsulated with positively charged nanoscale ZIF. It is possible to change the expression of the target gene of interest through efficient endosomal escape.

DNAs or nucleic acids encoding the negatively charged (1) and (2) may bind to cationic substances to form nanoparticles, which may penetrate into cells through receptor-mediated endocytosis or phagocytosis. The RNP complex of (1) and (2) may be bound to a cationic polymer. Examples of the cationic polymer include polyallylamine (PAH); polyethyleneimine (PEI); poly(L-lysine) (PLL); poly(L-arginine) (PLA); polyvinylamine homo- or copolymers; poly(vinylbenzyl-tri-C1-C4-alkylammonium salt); polymers of aliphatic or araliphatic dihalides and aliphatic N,N,N′,N′-tetra-C1-C4-alkyl-alkylenediamines; poly(vinylpyridine) or poly(vinylpyridinium salt); poly(N,N-diallyl-N,N-di-C1-C4-alkyl-ammonium halide); homo- or copolymers of quaternized di-C1-C4-alkyl-aminoethyl acrylates or methacrylates; POLYQUAD™; polyaminoamide, and the like.

Cationic lipids may include cationic liposome preparations. The liposomal lipid bilayer may protect the encapsulated nucleic acid from degradation and may prevent specific neutralization by antibodies capable of binding to the nucleic acid. During endosome maturation, the endosomal membrane and the liposome are fused together, allowing efficient endosomal escape of cationic lipid-nucleases. Examples of cationic lipids include polyethylenimine, poly(amidoamine) (PAMAM) starburst dendrimers, Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.). Exemplary cationic liposomes may be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N—(N′,N′-dimethylaminoethane)carbamoyl]cholesterol (DC-Chol), 2,3,-dioleyloxy-N-[2(sperminecarboxamido)ethyl]-N,N-dimethyl propanaminium trifluoroacetate (DOSPA), 1,2-dimyristyloxypropyl-3-dimethyl-hydroxyethyl ammonium bromide; or dimethyldioctadecylammonium bromide (DDRB).

With respect to the lipid nanoparticles, delivery can be achieved using a liposome as a carrier. The liposome is a spherical vesicle structure which is composed of single or multiple lamellar lipid bilayers surrounding internal aqueous compartments and an external, lipophilic phospholipid bilayer which is relatively impermeable. A liposome formulation may mainly contain natural phospholipids and lipids such as 1,2-distearolyl-sn-glycero-3-phosphatidyl choline (DSPC), sphingomyelin, phosphatidylcholine or monosialoganglioside. In some cases, cholesterol or 1,2-dioleoyl-sn-glycero-3-phosphoethanolamine (DOPE) may be added to the lipid membrane to eliminate plasma instability. Addition of cholesterol reduces rapid release of encapsulated bioactive compounds into the plasma or 1,2-dioleoyl-sn-glycero-3-phosphoethanolamine (DOPE) increases the stability.

In still another aspect, the present invention is directed to a genome editing method comprising a step of treating cells with the composition.

The cells are eukaryotic cells (e.g., cells derived from fungi such as yeast, eukaryotic animals and/or eukaryotic plants (e.g., embryonic cells, stem cells, somatic cells, germ cells, etc.)), cells derived from eukaryotic animals (e.g., primates such as humans or monkeys, dogs, pigs, cows, sheep, goats, mice, rats, etc.), or cells derived from eukaryotic plants (e.g., algae such as green algae, corn, soybean, wheat, rice, etc.), without being limited thereto.

### EXAMPLES

Hereinafter, the present invention will be described in more detail with reference to examples. These examples are only for illustrating the present invention, and it will be apparent to those of ordinary skill in the art that the scope of the present invention is not to be construed as being limited by these examples.

### Example 1. Cleavage of Target Sequence Using Cas9, nCas9-D10A, or nCas9-H840A

FIG. 1a shows: information on a target sequence; predicted cleavage positions upon cleavage with Cas9, nCas9-D10A and nCas9-H840A in vitro (red arrowhead: cleavage position, blue: PAM sequence); and results predicted when examining whole genome sequencing (WGS) data by IGV.

gDNA of HAP1 cells was treated with each of Cas9 variants at 37° C. for 16 hours, and WGS results were checked. Referring to FIG. 1B, Cas9 and nCas9-D10A showed the same cleavage pattern as expected, but in the case of nCas9-H840A, partial cleavage occurred even in the target strand, contrary to expectations.

In order to confirm the cleavage experiment performed on gDNA again, an in vitro cleavage experiment was performed using a plasmid. Upon electrophoresis, a supercoiled plasmid remains in a linear form when both strands are cleaved and in an open circular form when one strand is cleaved. With a 6,030-bp plasmid, an open circular plasmid and a linear plasmid for comparison were constructed using Nt.BbvCI enzyme that cleaves one strand and Spel enzyme that cleaves both strands, respectively. Thereafter, the plasmids were treated with each of Cas9, nCas9-D10A and nCas9-H840A, and the form of each plasmid was observed. Referring to FIG. 1c, it was confirmed that, when the plasmids were treated with Cas9, most of the plasmids were cleaved in both strands and remained in a linear form, and when the plasmids were treated with nCas9-D10A, most of the plasmids were cleaved in one strand and remained in an open circular form. However, it was confirmed that, when nCas9-H840A was used, more linear plasmids appeared than when nCas9-D10A was used. As a result of measuring the intensities of the bands using ImageJ software and obtaining relative linear band intensity values, it was observed that the relative band intensity values were linear 16.0% for nCas9-D10A and 43.3% for nCas9-H840A.

### Example 2. Examination of Indel (Insertion and Deletion) Frequency

As nuclease domains of SpCas9, an HNH domain and a RuvC domain exist, which cleave target DNA and non-target DNA, respectively. When mutation is introduced into the HNH domain or RuvC domain of Cas9, it is possible to produce a Cas9 nickase that can cut only one strand. As Cas9 nickase, a form in which D10A mutation is introduced into the RuvC domain or a form in which H840A or N863A mutation is introduced into the HNH domain is mainly used. As shown in FIG. 2a, mutations were introduced at positions D839, H840, N854 and N863 in the HNH domain, which are involved in DNA cleavage, to create a Cas9 nickase that can completely cut only a non-target strand.

To examine the frequency of unwanted indels (insertions and deletions) that can be introduced in cells by nickase Cas9 (nCas9), nCas9 was delivered into HEK293T cells together with plasmids expressing sgRNAs targeting various genes. Next, the cell DNA was isolated and analyzed by targeted deep-sequencing. Referring to FIG. 2b, it was confirmed that an indel frequency of 0.035 to 15% (2.5% on average) was shown by HNHv1(Cas9-H840A) which has been mainly used in the prior art. To reduce the indels, Cas9 variants having mutations of combinations of D839A, H840A, N854A and N863A in the Cas9 HNH domain were produced and used. As a result, it could be confirmed that the frequency of unwanted indels was reduced to less than 1% on average upon the use of various variants (HNHv5(H840A/N863A), HNHv7(H840A/N854A), HNHv9(N863A/N854A), HNHv11(H840A/N863A/N854A), HNHv12(H840A/D839A/N854A), HNHv13(N863A/D839A/N854A), and HNHv14(H840A/N863A/D839A/N854A)).

In order confirm whether the reduction in the frequency of unwanted indels as shown in the previous experiment is because the Cas9 variant is 1) a nickase form that accurately cuts only one strand or 2) a catalytically dead Cas9 form that lacks the activity of Cas9 and does not cut both strands, a double nicking experiment (an experiment using two sgRNAs that cut different strands) was conducted. In the case of 1), upon treatment with sgRNA-A or sgRNA-1, indels will not be observed, and upon treatment with both sgRNA-A and sgRNA-1, both strands will be cut (DNA double strand breaks) and indels will be observed. In the case of 2), indels will not be observed upon treatment with sgRNA-A, sgRNA-1, or sgRNA-A+sgRNA-1. As shown in FIG. 2c, as a result of confirming this prediction experimentally for two target sites, it could be confirmed that HNHv7, HNHv11, HNHv12 and HNHv14 all showed an indel frequency of 1% or less upon treatment with sgRNA-A, sgRNA-1, or sgRNA-A+sgRNA-1, suggesting that they are catalytically dead Cas9s that have lost almost all activity. On the other hand, it could be confirmed that HNHv5, HNHv9 and HNHv13 showed an indel frequency of 1% or less upon treatment with sgRNA-A or sgRNA-1, but showed an indel frequency of 1% or more upon treatment with both sgRNA-A and sgRNA-1, suggesting that they are in the form of a Cas9 nickase that cuts one strand.

### Example 3. Examination of Changes in Cleavage Patterns in In Vitro Experiment

gDNA of isolated cells was treated with each of nCas9-H840A and nCas9-H840A/N863A, and changes in the cleavage pattern of the gDNA were examined by WGS. As shown in FIG. 3a, as a result of targeting three different sites (HEK4, EMX1 and RUNX1), it could be confirmed that nCas9-H840A induced partial double strand cleavage, whereas, upon treatment with nCas9-H840A/N863A, cleavage of only a desired non-target strand occurred.

Pattern changes in the whole genome were examined by digenome sequencing. Digenome sequencing is one of the methods that can detect double-strand breaks in the whole genome. The patterns of double-strand breaks appearing in the whole genome were compared through digenome sequencing, and the results were displayed by Circos plots. Referring to FIG. 3b, when three different sites (HEK4, EMX1 and RUNX1) were treated with nCas9-H840A, double-strand breaks were observed at the target sites and off-target sites. On the other hand, upon treatment with nCas9-H840A/N863A, double-stranded breaks could not be observed at the target sites, and it could be confirmed that double strand breaks at off-target sites disappeared or the percentage thereof was significantly reduced. Thereby, it was confirmed from the in vitro experimental results that Cas0-H840A/N863A is a nickase Cas9 form that can cut only one strand of DNA, as shown in FIG. 1.

### Example 4. Examination of Gene Editing Efficiency and Indel Frequency

A prime editor (PE) composed of nCas9 and MMLV reverse transcriptase was delivered to cells together with pegRNA capable of inducing a mutation to be introduced, and DNA was analyzed by targeted deep-sequencing. The results are shown in FIG. 4.

As shown in FIGS. 4a and 4B, the efficiency of desired gene editing (correct editing) (a) and the unwanted indel activity (frequency) (b) were measured. c and d show non-normalized NGS data for (a)/(b).

The indicated values were all normalized to 1, which is a value for conventional PEv1(PE-H840A). When the efficiency of desired gene editing is higher than 1, it is shown in pink, and when the efficiency of desired gene editing is lower than 1, it is shown in green. When the unwanted indel activity is higher than 1, it is shown in red, and when the unwanted indel activity is lower than 1, it is shown in blue.

(a, c) When PE variants were prepared using the Cas9 variants used in FIG. 1 and were tested, it can be seen that, in the case of PE-HNHv3, PE-HNHv5, PE-HNHv6 and PE-HNHv8, PE-HNHv10 in comparison with conventional PE-HNHv1(PE2-H840A), the correct editing efficiency was retained. (Since it is preferable that the desired editing efficiency is not reduced, the values in FIG. 4a should not be green.)

(b, d) It can be confirmed that the frequency of unwanted indels introduced by PE-HNHv1 was reduced to less than half when PE-HNHv5, PE-HNHv7, PE-HNHv9, PE-HNHv11, PE-HNHv12, PE-HNHv13 or PE-HNHv14 was used. (Since it is preferable that the frequency of unwanted indels be reduced, it is preferable that the values in FIG. 4b are blue.)

Thereby, it was confirmed that, when PE-HNHv5 (PE2-H840A/N863A) among the HNH domain variants of PE obtained by introducing mutations into the HNH domain was used, the frequency of unwanted indels was reduced compared to when the conventional PE-HNHv1(PE2-H840A) was used, and the desired genome editing efficiency was retained. In addition, it could be confirmed that, even when PE2-Cas9-WT composed of a Cas9 nuclease form (the form in which the conventional H840A mutation was removed) was used, the desired genome editing efficiency of 13.0% on average was obtainable, and in targets in which the efficiency of PE is very low, the correct editing efficiency was sometimes increased when Cas9 nuclease was used (the pink color observed in the PE-Cas9-WT portion in FIG. 4a).

### Example 5. Examination of Gene Editing Efficiency and Unwanted Indel Frequency Upon Use of Cas9 Variants Containing Deletion Mutations

Gene editing efficiency and unwanted indel frequency upon the use of Cas9 variants containing a deletion of additional amino acid residues were examined.

To further reduce unwanted indel mutations, HNH deletion variants (HNHΔ1 to 12) were prepared by deleting a portion of the HNH domain of Cas9 and then linking with linkers of various lengths (amino acid sequences: AS, GGGGS, and GGGGSGGGGS) (FIG. 5a).

The frequency of unwanted indels introduced into various HNH deletion variants (HNHΔ1 to 12) obtained by introducing HNH deletion into Cas9 was measured at three different target sites. Referring to FIG. 5b, it was confirmed that Cas9-HNHΔ1 to 12 introduced indels with much lower efficiency than the conventional Cas9-H840A and the Cas9-HNHv5(Cas9-H840A/N863A) identified in the previous experiment.

PE variants were prepared using various HNH deletion variants (HNHΔ1 to 12), and cells were treated with the PE variants. Then, the efficiency of correct genome editing was measured by targeted deep-sequencing. Referring to FIG. 5c, it was confirmed that, in the case of PE-HNHΔ4 to 9, desired editing occurred well with similar efficiency or half efficiency compared to that in the case of the conventional PE or PE-HNHv5.

The frequency of unwanted indels that can be introduced by PE-HNH deletion variants (HNHΔ1 to 12) was measured. Referring to FIG. 5d, it was confirmed that, in the case of PE-HNHΔ4 to 9, unwanted indels were significantly reduced. In addition, it was confirmed that the frequency of unwanted indels was reduced compared to when the previous HNH point mutation variants (HNHv1 to 14) were used. Thereby, it was confirmed that, when PEs without the 792-897 amino acid portion or 786-885 amino acid portion of Cas9 are used, the introduction of unwanted indels may be reduced and correct gene editing may occur well. As a result, even if about 100 amino acids in the Cas9 sequence are deleted, the gene editing function of PEs can be performed well, and the sizes of Cas9 and PE proteins also become smaller.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### SEQUENCE LIST FREE TEXT

Electronic file attached.

1. A nuclease variant or a nucleic acid encoding the same, in which one or more amino acids selected from the group consisting of D839, H840, N854 and N863 in the sequence of SEQ ID NO: 1 are substituted with other amino acid(s).

2. The nuclease variant or nucleic acid encoding the same according to claim 1, wherein the nuclease variant contains one or more mutations selected from the group consisting of the following mutations:
a substitution of alanine for D839 in the sequence of SEQ ID NO: 1;
a substitution of alanine for H840 in the sequence of SEQ ID NO: 1;
a substitution of alanine for N854 in the sequence of SEQ ID NO: 1; and
a substitution of alanine for N863 in the sequence of SEQ ID NO: 1.

3. The nuclease variant or nucleic acid encoding the same according to claim 1, wherein the nuclease variant comprise a sequence selected from the group consisting of SEQ ID NOs: 2 to 15.

4. A nuclease variant or a nucleic acid encoding the same, the nuclease variant containing a deletion of one or more amino acid residues selected from the group consisting of the following amino acid residues in a sequence selected from the group consisting of SEQ ID NOs: 1 to 15:
a deletion of one or more amino acid residues at positions 824 to 874 in a sequence selected from the group consisting of SEQ ID NOs: 1 to 15;
a deletion of one or more amino acid residues at positions 792 to 897 in a sequence selected from the group consisting of SEQ ID NOs: 1 to 15;
a deletion of one or more amino acid residues at positions 786 to 885 in a sequence selected from the group consisting of SEQ ID NOs: 1 to 15; and
a deletion of one or more amino acid residues at positions 765 to 908 in a sequence selected from the group consisting of SEQ ID NOs: 1 to 15.

5. The nuclease variant or nucleic acid encoding the same according to claim 4, wherein the nuclease variant comprises a sequence selected from the group consisting of SEQ ID NOs: 16 to 19.

6. A method for genome editing comprising a step of treating cells with a composition containing:
(1) a prime editor protein comprising a nuclease or a variant thereof and a reverse transcriptase, or a nucleic acid encoding the prime editor protein; and
(2) a prime editing guide RNA (pegRNA) comprising a binding site, which binds to a genome to be edited, and an editing sequence.

7. The method of claim 6, wherein the nuclease is Cas9.

8. The method of claim 6, wherein the nuclease variant is one in which one or more amino acids selected from the group consisting of D839, H840, N854 and N863 in the sequence of SEQ ID NO: 1 are substituted with other amino acid(s).

9. The method of claim 6, wherein the nuclease variant contains one or more mutations selected from the group consisting of the following mutations:
a substitution of alanine for D839 in the sequence of SEQ ID NO: 1;
a substitution of alanine for H840 in the sequence of SEQ ID NO: 1;
a substitution of alanine for N854 in the sequence of SEQ ID NO: 1; and
a substitution of alanine for N863 in the sequence of SEQ ID NO: 1.

10. The method of claim 6, wherein the nuclease variant comprises a sequence selected from the group consisting of SEQ ID NOs: 2 to 15.

11. The method of claim 6, wherein the nuclease variant contains a deletion of one or more amino acid residues selected from the group consisting of the following:
a deletion of one or more amino acid residues at positions 824 to 874 in a sequence selected from the group consisting of SEQ ID NOs: 1 to 15;
a deletion of one or more amino acid residues at positions 792 to 897 in a sequence selected from the group consisting of SEQ ID NOs: 1 to 15;
a deletion of one or more amino acid residues at positions 786 to 885 in a sequence selected from the group consisting of SEQ ID NOs: 1 to 15; and
a deletion of one or more amino acid residues at positions 765 to 908 in a sequence selected from the group consisting of SEQ ID NOs: 1 to 15.

12. The method of claim 11, wherein the nuclease variant comprises a sequence selected from the group consisting of SEQ ID NOs: 16 to 19.

13. The method of claim 11, further containing a peptide linker.

14. The method of claim 13, wherein the linker is (AnS)m (where n and m are each 1 to 10), (GS)n, (GGS)n, (GSGGS)n, or (GnS)m (where n and m are each 1 to 10).

15. The method of claim 6, wherein the nuclease or variant thereof or the reverse transcriptase are contained individually or in the form of a fusion protein.

16. The method of claim 6, wherein the reverse transcriptase is derived from M-MLV (Moloney murine leukemia virus).

17. The method of claim 6, wherein the reverse transcriptase comprises the sequence of SEQ ID NO: 29.

18. The method of claim 6, containing a vector which contains the nucleic acid encoding the prime editor protein and a nucleic acid encoding the prime editing guide RNA either individually or in a complex form.

19. The method of claim 6, containing a vector which contains nucleic acids encoding the prime editor protein and the prime editing guide RNA.

20. (canceled)


SEQUENCE LISTING (29 sequences)

SEQ ID NO: 1 - protein, 1368 aa, Artificial Sequence [sequence omitted]

SEQ ID NO: 2 - protein, 1368 aa, Artificial Sequence
  HNHv1_H840A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1368 aa, Artificial Sequence
  HNHv2_N863A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 1368 aa, Artificial Sequence
  HNHv3_D839A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1368 aa, Artificial Sequence
  HNHv4_N854A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDAKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 1368 aa, Artificial Sequence
  HNHv5_H840A/N863A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - protein, 1368 aa, Artificial Sequence
  HNHv6_H840A/D839A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - protein, 1368 aa, Artificial Sequence
  HNHv7_H840A/N854A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDAKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - protein, 1368 aa, Artificial Sequence
  HNHv8_N863A/D839A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 10 - protein, 1368 aa, Artificial Sequence
  HNHv9_N863A/N854A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDAKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - protein, 1368 aa, Artificial Sequence
  HNHv10_H840A/N863A/D839A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - protein, 1368 aa, Artificial Sequence
  HNHv11_H840A/N863A/N854A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDAKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 13 - protein, 1368 aa, Artificial Sequence
  HNHv12_H840A/D839A/N854A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDAKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 14 - protein, 1368 aa, Artificial Sequence
  HNHv13_N863A/D839A/N854A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAHIVPQSFLKDDSIDAKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 15 - protein, 1368 aa, Artificial Sequence
  HNHv14_H840A/N863A/D839A/N854A
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDAKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 16 - protein, 1317 aa, Artificial Sequence
  HNH1˜3_824-874
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 17 - protein, 1262 aa, Artificial Sequence
  HNH4˜6_792-897
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 18 - protein, 1268 aa, Artificial Sequence
  HNH4˜6_786-885
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIELLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 19 - protein, 1224 aa, Artificial Sequence
  HNH7˜9_765-908
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMASELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 20 - DNA, 23 bp, Artificial Sequence
  Target Sequence
gatgtctgcaggccagatgaggg

SEQ ID NO: 21 - DNA, 23 bp, Artificial Sequence
  Target Sequence
ccctcatctggcctgcagacatc

SEQ ID NO: 22 - DNA, 57 bp, Artificial Sequence
  Target Sequence
gcctgagtccgagcagaagaagaagggctcccatcacatcaaccggtggcgcattgc

SEQ ID NO: 23 - DNA, 57 bp, Artificial Sequence
  Target Sequence
gcaatgcgccaccggttgatgtgatgggagcccttcttcttctgctcggactcaggc

SEQ ID NO: 24 - DNA, 74 bp, Artificial Sequence
  Target Sequence
tgcattttcaggaggaagcgatggcttcagacagcatatttgagtcatttccttcgtacccacagtgcttcatg

SEQ ID NO: 25 - DNA, 74 bp, Artificial Sequence
  Target Sequence
catgaagcactgtgggtacgaaggaaatgactcaaatatgctgtctgaagccatcgcttcctcctgaaaatgca

SEQ ID NO: 26 - DNA, 23 bp, Artificial Sequence
  Target Sequence
ggcactgcggctggaggtggggg

SEQ ID NO: 27 - DNA, 23 bp, Artificial Sequence
  Target Sequence
gagtccgagcagaagaagaaggg

SEQ ID NO: 28 - DNA, 23 bp, Artificial Sequence
  Target Sequence
ccatcgcttcctcctgaaaatgc

SEQ ID NO: 29 - protein, 677 aa, Artificial Sequence
  MMLV-RT
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP